(19) United States
(12) Patent Application Publication
(10) Pub. No.: US 2010/0234454 A1
(43) Pub. Date: Sep. 16, 2010

(54) TREATMENT OF CNIDARIA INTOXICATION

(75) Inventors: Eva Cuypers, Heist-op-den-Berg (BE); Evert Karlsson, Uppsala (SE); Jan Tytgat, Lubbeek (BE); Angel Yanagihara, Manoa, HI (US)

Correspondence Address: CLARK & ELBING LLP, 101 FEDERAL STREET, BOSTON, MA, 02110, US

(73) Assignees: Katholieke Universiteit Leuven K.U. Leuven R&D, Leuven (BE); University of Hawaii, Honolulu (US)

(21) Appl. No.: 12/304,047

(22) PCT Filed: Jun. 11, 2007
(86) PCT No.: PCT/BE07/00056

§ 371 Date: Aug. 27, 2009

(30) Foreign Application Priority Data
Jun. 9, 2006 (GB) 0611417.7

(51) Int. Cl. A61K 31/165; C07D 223/16; C07D 317/70; C07C 233/19; C07K 2/00; C07J 17/00; C07C 233/16; A61K 31/357; A61P 43/00

(52) U.S. Cl. 514/463; 540/593; 549/432; 564/192; 530/300; 536/5; 548/504; 554/66; 514/627; 514/625

(57) ABSTRACT

The present invention relates to the use of vanilloid receptor (VR) antagonists, and more particularly vanilloid receptor 1 (VR1) antagonist, as analgesics in the treatment and/or prohylaxis of cnidaria envenomations.

### FIELD OF THE INVENTION

The present invention relates to the use of antagonist of the non-selective cation channel TRPV1 as a medicine for the treatment of Cnidaria intoxication.

### BACKGROUND OF THE INVENTION

Every year hundred of thousands of people worldwide are stung by cnidaria. These envenomations, which are characterised by local erythrema, burning pain, hypersensitivity, dermonecrosis and sometimes even death¹⁻³, represent a major cost in terms of human suffering and economic loss. Currently, clinical management merely attempts to address secondary symptoms, such as pain and inflammation. Inadequate understanding of the primary pathofysiological pathways initiated by envenomation is a major impediment to design more effective treatments. Empirical partial post-envenomation pain relief has been associated with topical vinegar application or hot-water immersion⁴⁻⁶. Since some members of the TRP super family are known to be heat and pH sensitive⁷⁻¹², we tested whether TRPV1 is involved in cnidarian envenomation.

The capsaicin receptor gene was cloned in 1997. It was presumed from its amino acid sequence that it was an ion channel having a six-transmembrane domain. Since capsaicin has a vanillyl group in the structure, it is generically referred to as vanilloids along with its analogs such as RTX, and the cloned receptor was named vanilloid receptor subtype 1, referred to as VR1; This VR1 may be also referred to as TPRV1 (transient receptor potential vanilloid receptor 1)). Then, electrophysiological functional analysis using the voltage clamp or patch clamp method has been performed respectively by making oocytes of Xenopus laevis or human derived cultured cells to express VR1, and it has been revealed that VR1 is directly activated by capsaicin, without mediated by an intracellular second messenger (see, for example, Szallasi A, Blumberg P M. (1999) Pharmacol. Rev. 51, 159-212), and that VR1 is anon-selective cation ion channel having high Ca²⁺ permeability with an outward rectification property (see, for example, Premkumar L S, Agarwal S, Steffen D. (2002) J. Physiol. 545, 107-117).

### SUMMARY OF THE INVENTION

The present invention is based on the surprising finding that the burning bain sensation associated with cnidaria envenomation is at least in part due to an allosteric mechanism in which the desensitization is knocked down by the venom. Additionally, it was shown that vanilloid receptor (VR) antagonists, and more particularly vanilloid receptor 1 (VR1) antagonist, can be used as analgesics in the treatment and/or prohylaxis of cnidaria envenomations. Therefore, in a first embodiment the present invention relates to the use of a vanilloid receptor antagonist, preferably a VR1 antagonist, or a pharmaceutically acceptable derivative thereof, in the manufacture of a medicament for the treatment and/or prophylaxis of cnidaria envenomations and the pain associated therewith.

In a second object the present invention provides a method for the treatment and/or prophylaxis of cnidaria envenomations and the pain associated therewith, said method comprising the administration of an effective, non-toxic and pharmaceutically acceptable amount of a vanilloid receptor antagonist, preferably a VR1 antagonist, or a pharmaceutically acceptable derivative thereof.

In a third object the present invention provides a pharmaceutical composition for the treatment and/or prophylaxis of cnidaria envenomations and the pain associated therewith, which composition comprises a vanilloid receptor antagonist, preferably a VR1 antagonist, or a pharmaceutically acceptable derivative thereof.

In a fourth object the present invention provides the use of a vanilloid receptor antagonist, preferably a VR1 antagonist, or a pharmaceutically acceptable derivative thereof in the treatment and/or prophylaxis of cnidaria envenomations and the pain associated therewith.

### DESCRIPTION

### List of Figures

FIG. 1. Effects of Cnidaria and BmK venom on TRPV1.

Traces from A. Aiptasia pulchella (A.P.) venom B. Chironex fleckeri (C.F.) venom C. Physalia physalis (P.P) venom D. Cyanea capillata (C.C.) venom For A-D Left: Crude venom in the absence of capsaicin (CAP) and capsazepine (CZP). Right: Allosteric effect of venom with 2 μM capsaicin. E. Left: Trace with lack of effect of BmK venom in the presence of 2 μM capsaicin. Right: Current-voltage relation (IV) showing the effect of ND96, capsaicin and BmK added to capsaicin. F. Allosteric effect of Cyanea capillata venom and anandamide (ANA).

FIG. 2. TRPV1 desensitization knocked down by Cyanea capillata venom.

A. TRPV1 current during the first activation. The venom is added on the peak of this activation. B. Current during the first activation when the venom is added just at the time when desensitization is macroscopically visible. It can be seen that the venom quickly knocks down desensitization (see arrow). C. Trace showing the first and second activation of the channel and the effect of the venom when added during the second activation. Note that the venom restores exactly the desensitization-dependent fraction of the inward current (see arrow).

FIG. 3. In vivo effect of Cyanea capillata with and without TRPV1 antagonist.

A. Number of flinches during 4 minutes observation period. Effect after left-hindpaw plantar injection of capsaicin (black) and vehicle of capsaicin (left diagonals). Pretreatment by subcutanous injection of 40 mg/kg BCTC (white) or vehicle (right diagonals) and one hour later the number of flinches and bites were observed after an intraplantar injection of capsaicin (n=6). The effect of capsaicin with BCTC was significantly different from the effect of capsaicin alone. The BCTC vehicle did not give a significant difference as compared with capsaicin alone. B. Same tests as in A. Here the effect of the venom is tested instead of capsaicin. The effect of venom with BCTC was significantly different from the effect with venom alone. The BCTC vehicle did not give a significant difference as compared with venom alone.

### DESCRIPTION

The present invention is based on the surprising finding that the burning pain sensation associated with cnidaria envenomation is at least in part due to an allosteric mechanism in which the desensitization is knocked down by the venom. Additionally, it was shown that vanilloid receptor (VR) antagonists, and more particularly vanilloid receptor 1 (VR1) antagonist, can be used as analgesics in the treatment and/or prophylaxis of cnidaria envenomations. Therefore, in a first object the present invention provides the use of VR receptor antagonists and more particular VR1 receptor antagonists in the manufacture of a medicine for the treatment and/or prophylaxis of cnidaria envenomations.

Suitable vanilloid receptor antagonists for use in accordance with the present invention include those disclosed in European Patent numbers EP 0 347 000 and EP 0 401 903; UK Patent Application Number GB2226313; International Patent Applications, Publication Numbers WO 92/09285, WO 01/021577, WO 02/08221, WO 02/16317, WO 02/16318, WO 02/16319, WO 02/072536, WO02/090326, WO 03/022809, WO 03/053945, WO03097586, WO03070247, WO03080578, WO030055484, WO03068749, WO03095420, WO04002983, WO02076946, WO04033435, WO2006038041, WO2007050732, WO2005123666, WO2006122799, WO2003099284, WO200611346, DE 102005044814, WO2006072736, WO2006045498, WO2007054474 and WO03062209; International Patent Application Number PCT/GB03/00608; and U.S. Pat. Nos. 3,424,760, 3,424,761, US20040157849, US20040209884, US20050113576, US20040254188, US20050043351, US20050085512, US20040138454, US20050107388, US20050187291, US20050154230, US20050049241, US2007099954 and US20060035939. Suitable vanilloid receptor antagonists for use in accordance with the present invention further include those disclosed in the Journal of medicinal Chemistry, 2005, 48, pages 5823-5836, Journal of medicinal Chemistry, 2005, 48, pages 744-752, Journal of medicinal Chemistry, 2005, 48, pages 1857-1872, Journal of medicinal Chemistry, 2005, 48, pages 4663-4669, Journal of medicinal Chemistry, 2005, 48, pages 71-90, Bioorganic & medicinal chemistry letters, 2001, 9, pages 1713-1720, J. Med. Chem.; 2005; 48(1) pages 71-90, Mol Pharmacol, 2005, 68, pages 1524-1533, Bioorganic & medicinal chemistry letters, 2001, 9, pages 631-634 and Recent Patents on CNS Drug Discovery, 2006, 1, pages 65-76, Bioorganic & Medicinal Chemistry Letters(2006) 5217-5221, Bioorganic & Medicinal Chemistry Letters(2007), 214-219, British Journal of Pharmacology (2007) 150, 766-781 and JPET (2007) 321: 791-798. The above cited patents, patent applications and references are included herein by reference.

Compounds suitable to be used as VR1 antagonist can be selected out of compounds derived from the TRPV1 agonists capsaicin, resiniferatoxin, nordihydrocapsaicin, nonivamide, arvanil and phenacetylrinvanil. Examples of such compounds are capsazepine, iodo-resiniferatoxin and halogenated, more particularly iodinated derivatives of nordihydrocapsaicin, nonivamide, arvanil and phenacetylrinvanil. An other class of suitable TRPV1 antagonist are selected out of the groups respectively comprising fused azabicyclic compounds (as disclosed in US20050113576), fused heterocyclic compounds (as disclosed in US20040254188), amide compounds (as disclosed in US20050085512) and fused Pyridine derivatives (as disclosed in US20040138454). Furthermore, WO2003099284 discloses amino-pyridine, -pyridine and pyridazine derivatives for use as vanilloid receptor ligands. Another structural class of TRPV1 anatagonists is the pyridyl piprazyl ureas, comprising amongst others piperazine-1-carboxanilidines and pyridazinylpiperazines and the compounds disclosed in US20050049241 having TRPV1 antagonistic activity. Also other urea and thiourea derivatives have been identified as TRPV1 antagonists, such as heteroaromatic urea derivatives (as disclosed in US20050107388), beta-aminotetralin-derived urea compounds (as disclosed in US200501087291), isoquinoline urea derivatives (as disclosed in J Pharmacol Exp Ther 2005; 314: 400-9), arylureas, biarylureas (bioorganic & Medicinal Chemistry Letters, 2006, p 5217-5221), N-(4-chlorobenzyl)-N′-(4-hydroxy-3iodo-5-methoxybenzylthiourea (JPET (2007) 321:791-798), the urea and indazole derivatives having TRPV1 antagonistic activity disclosed in US20050154230 and WO2007050732, respectively. WO2005123666 also discloses selected urea and thiourea derivatives with TRPV1 antagonistic activity, while WO2006111346 describes substituted cyclic urea derivatives as TRPV1 modulating compounds. Recently, US2007099954 disclosed prodrugs of urea containing compounds for use as a TRPV1 antagonist.

Furthermore, several cinnamide derivatives have been reported as potent TRPV1 antagonists, for example (E)-3-(4-t-Butylphenyl)-N-(2,3-dihydrobenzo[b][1,4]dioxin-6-yl)acrylamide (AMG 9810; Journal of Pharmacology And Experimental Therapeutics, 2005, 313, pages 474-484) and N-(3-methoxyphenyl)-4-chlorocinnamide (SB-366791, Neuropharmacology 2004; 46, pages 133-49). TRPV1 antagonistic activity was also reported for some arginine rich peptides, ginsenosides, Terpens, isovelleral, aminoquinazolines, N-arachidonoyl-serotonin, oleoylethanolamide and Methanandamide. Patent application WO2006038041 provides besylate salts of six-membered amino-heterocycles, which can be used as vanilloid-1 receptor antagonists, said compounds having the general formula: Y-J-NH—Z (I) wherein: Y is a quinoline or isoquinoline optionally substituted with h one or two substituents independently chosen from hydroxy, halogen, haloC 1-4 alkyl, C 1-4 alkyl, C 1-4 alkoxy, haloC 1-4 alkoxy, nitro and amino; J is pyridine, pyridazine, pyrazine, pyrimidine or triazine optionally substituted with one or two substituents in dependently chosen from hydroxy, halogen, haloC 1-4 alkyl, C 1-4 alkyl, C 3-5 cycloalkyl, C 1-4 alkoxy, hydroxyC 1-4 alkyl, cyano, hydroxy, C 1-4 cycloalkoxy, C 1-4 alkylthio, haloC 1-4 alkoxy, nitro, Q, (CH 2) p Q, —NR 2 R 3, —(CH 2) p NR 2 R 3 and —O(CH 2) p NR 2 R 3; wherein J is substituted at positions meta to each other by NH and Y; and Z is phenyl or pyridyl optionally substituted with one or two substituents independently selected from halogen, haloC 1-4 alkyl, C 1-4 alkyl, C 1-4 alkoxy, haloC 1-4 alkoxy, nitro and amino; Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by C 1-4 alkyl; each R 2 and R 3 is chosen from H and C 1-4 alkyl, or R 2 and R 3, together with the nitrogen atom to which they are attached, may form a six-membered ring optionally containing an oxygen atom or a further nitrogen atom, which ring is optionally substituted by C 1-4 alkyl or Q; p is 1, 2 or 3. WO2006122200 discloses 2,3-substituted fused bicyclic pyrimidin-4(3H)-ones as TRPV1 modulating agents.

Patent application WO2006122799 discloses substituted benzo[d]isoxazol-3-yl-amine compounds having a high affinity for TRPV1. DE102005044814 discloses spiro-isoxazole-cycloalkane compounds and WO2006072736 provides N-(heteroaryl)-1H-indole-2-carboxamide derivatives having TRPV1 inhibitory activity. Furthermore, WO2006045498 provides sulfonamido compounds that antagonise the vanilloid TRPV1 receptor. More recently WO2007054480 and WO2007054474 and WO200705447 disclosed 2-(benzimidazol-1-YL)-acetamide biaryl derivatives and 2-(benzimidazol-1-yl)-N-(4-phenylthiazol-2-yl) acetamide derivatives for use as TRPV1 inhibitors.

Table 1 presents the structures of known VR 1 antagonists. The use of these compounds or pharmaceutically acceptable derivatives thereof are preferred for use in accordance with the present invention include those in table 1. The references cited in table 1 are included herein by reference. The R-groups as presented in the structures of table 1 are independently selected from the group consisting of hydrogen; C1-18 alkyl (including haloalkyl), preferably C1-6 alkyl; C2-18 alkenyl; C2-18 alkynyl; C1-18 alkoxy, preferably C1-6 alkoxy; C1-18 alkylthio; C3-10 cycloalkyl; C4-10 cycloalkenyl; C4-10 cycloalkynyl; halogen; —OH; —SH; —CN; —NO2; —NZ2Z3; —OCF3; C(═O)Z4; C(═S)Z4; aryl; aryloxy; arylthio; arylalkyl; heterocycle; oxyheterocycle; thioheterocycle; and each of said alkyl, alkenyl, alkynyl, alkoxy, alkylthio, cycloalkyl, cycloalkenyl, cycloalkynyl, aryl, aryloxy, arylthio, arylalkyl, heterocycle; oxyheterocycle; thioheterocycle can be substituted with 1 or more Z1;
Z1 is independently selected from the group consisting of hydrogen; C1-18 alkyl (including haloalkyl), preferably C1-6 alkyl; C2-18 alkenyl; C2-18 alkynyl; C1-18 alkoxy, preferably C1-6 alkoxy; C1-18 alkylthio; C3-10 cycloalkyl; C4-10 cycloalkenyl; C4-10 cycloalkynyl; halogen; OH; SH; CN; NO2; —NZ2Z3; —OCF3; C(═O)Z4; C(═S)Z4; aryl; aryloxy; arylthio; arylalkyl; heterocycle; oxyheterocycle; thioheterocycle;
each Z2 and Z3 is independently selected from hydrogen; C1-18 alkyl, preferably C1-6 alkyl; aryl, preferably phenyl; and C(═O)Z5;
Z4 is selected from hydrogen; OH; C1-18 alkyl; C1-18 alkoxy; NZ2Z3; aryl;
Z5 is selected from hydrogen; OH; C1-18 alkyl; C1-18 alkoxy; aryl.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “C1-18 alkyl” as used herein means C1-C18 normal, secondary, or tertiary hydrocarbon. Examples are methyl, ethyl, 1-propyl, 2-propyl, 1-butyl, 2-methyl-1-propyl(i-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, cyclopropyl, cyclobutyl, cyclopentyl and cyclohexyl. The term also includes C1-18 haloalkyl, which is a C1-18 alkyl bearing at least one halogen.

As used herein and unless otherwise stated, the term “C3-10 cycloalkyl” means a monocyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C7-10 polycyclic saturated hydrocarbon monovalent radical having from 7 to 10 carbon atoms such as, for instance, norbornyl, fenchyl, trimethyltricycloheptyl or adamantyl.

The terms “C2-18 alkenyl” and “C3-10 cycloalkenyl” as used herein is C2-C18 normal, secondary or tertiary and respectively C3-10 cyclic hydrocarbon with at least one site (usually 1 to 3, preferably 1) of unsaturation, i.e. a carbon-carbon, sp2 double bond. Examples include, but are not limited to: ethylene or vinyl (—CH═CH2), allyl (—CH2-CH═CH2), cyclopentenyl (−C5H7), and 5-hexenyl (—CH2 CH2CH2CH2CH═CH2). The double bond may be in the cis or trans configuration.

The terms “C2-18 alkynyl” and “C3-10 cycloalkynyl” as used herein refer respectively to C2-C18 normal, secondary, tertiary or the C3-C10 cyclic hydrocarbon with at least one site (usually 1 to 3, preferably 1) of unsaturation, i.e. a carbon-carbon, sp triple bond. Examples include, but are not limited to: acetylenic (—C≡CH) and propargyl (—CH2C≡CH).

The term “aryl” as used herein means a aromatic hydrocarbon radical of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, spiro, anthracene, biphenyl, and the like.

“Arylalkyl” as used herein refers to an alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

The term “heterocycle” means a saturated, unsaturated or aromatic ring system including at least one N, O, S, or P. Heterocycle thus include heteroaryl groups. Heterocycle as used herein includes by way of example and not limitation these heterocycles described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. Examples of heterocycles include by way of example and not limitation pyridyl, dihydroypyridyl, tetrahydropyridyl. (piperidyl), thiazolyl, tetrahydrothiophenyl, sulfur oxidized tetrahydrothiophenyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, a carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl.

Heteroaryl includes by way of example and not limitation pyridyl, dihydropyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

By way of example, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicycle [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes some aryl groups.

As used herein and unless otherwise stated, the terms “C1-18 alkoxy”, “C3-10 cycloalkoxy”, “aryloxy”, “arylalkyloxy”, “oxyheterocycle”, “thio C1-7 alkyl”, “thio C3-10 cycloalkyl”, “arylthio, “arylalkylthio” and “thioheterocycle” refer to substituents wherein a C1-18 alkyl radical, respectively a C3-10 cycloalkyl, aryl, arylalkyl or heterocycle radical (each of them such as defined herein), are attached to an oxygen atom or a sulfur atom through a single bond, such as but not limited in methoxy, ethoxy, propoxy, butoxy, thioethyl, thiomethyl, phenyloxy, benzyloxy, mercaptobenzyl and the like.

As used herein and unless otherwise stated, the term halogen means any atom selected from the group consisting of fluorine, chlorine, bromine and iodine.

Any substituent designation that is found in more than one site in a compound of this invention shall be independently selected.

Particularly preferred vanilloid receptor antagonists for use in accordance to the present invention are N-(2-Bromophenyl)-N′-[((R)-1-(5-trifluoromethyl-2pyridyl)pyrrolidin-3-yl)]urea (WO03/022809), or a pharmaceutically acceptable derivative thereof; N-(4-Tertiarybutylphenyl)-4-(3-cholorphyridin-2-yl)tetrahydropyrazine-1(2H)-carbox-amide (BCTC) or a pharmaceutically acceptable derivative thereof (Journal of Pharmacology and Experimental Therapeutics, 306:387-393, 2003); the compound referred to as A-784168 (structure see Table 1), the compound referred to as SB-705498 (structure see Table 1), the compound referred to as GRC 6211 (Glenmark Pharmaceuticals, LTD),), the compound referred to as GRC 6211 (Neurogen Coporation), (N-1H-indazol-4-yl-N′-[(1R)-5-piperidin-1-yl-2,3-dihydro-1H-inden-1-yl]urea), and the compound referred to as AMG 517 (structure see Table 1)(Amgen).

All publications, including but not limited to patents and patent applications, cited in this specification are herein incorporated by reference as if each individual publication were specifically and individually indicated to be incorporated by reference herein as though fully set forth.

Certain vanilloid receptor antagonists may exist in one of several tautomeric forms, all of which are encompassed by the present invention as individual tautomeric forms or as mixtures thereof. Where a vanilloid receptor antagonist contains a chiral carbon, and hence exists in one or more stereoisomeric forms or where one or more geometric isomers exist, it will be appreciated that the method of the present invention encompasses all of the said forms of the vanilloid receptor antagonists whether as individual isomers or as mixtures of isomers, including racemates. When used herein the term ‘vanilloid receptor antagonist’ relates to an antagonist, such as a small molecular weight antagonist, of the vanilloid receptor. It will be appreciated that the term also embraces suitable pharmaceutically acceptable derivatives thereof.

Vanilloid receptor antagonist activity may be assessed by use of the methodologies disclosed in the applications, such as, WO 02/08221, WO 02/16317 and WO 02/090326, which, are included herein by reference.

Suitable pharmaceutically acceptable derivatives of a vanilloid receptor antagonist are, for example, salts and solvates.

Suitable pharmaceutically acceptable derivatives of any particular vanilloid receptor antagonist include those disclosed in the above-mentioned publications.

Suitable pharmaceutically acceptable salts include salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroebietylamine, N,N′-bisdehydroebietylamine, glucamine, N-methylglucamine or bases of the pyridine type such as pyridine, colliding, quinine or quinoline.

Suitable acid addition salts include pharmaceutically acceptable inorganic salts such as the sulfate, nitrate, phosphate, borate, hydrochloride and hydrobromide and pharmaceutically acceptable organic acid addition salts such as acetate, tartrate, maleate, citrate, succinate, benzoate, ascorbate, methane sulfonate, α-keto glutarate and α-glycerophosphate, especially the maleate salt.

The vanilloid receptor antagonists referred to herein are conveniently prepared according to the methods disclosed in the above mentioned patent publications in which they are disclosed.

The salts and/or solvates of the vanilloid receptor antagonists referred to herein may be prepared and isolated according to conventional procedures for example those disclosed in the above mentioned patent publications.

The present invention also provides a vanilloid receptor antagonist or a pharmaceutically acceptable derivative thereof, for use in the treatment and/or prophylaxis of cnidaria envenomations and pain associated therewith.

The present invention also provides a vanilloid receptor antagonist or a pharmaceutically acceptable derivative thereof, for use in a method for the treatment and/or prophylaxis of cnidaria envenomations and pain associated therewith.

In the above-mentioned method the vanilloid receptor antagonist, may be administered per se or, preferably, as a pharmaceutical composition also comprising a pharmaceutically acceptable carrier.

In the treatment of the invention, the vanilloid receptor antagonist mentioned herein is formulated and administered in accordance with the methods disclosed in the above mentioned patent applications and patents.

Accordingly, the present invention also provides a pharmaceutical composition for the treatment and/or prophylaxis of cnidaria envenomations and pain associated therewith, which composition comprises a vanilloid antagonist, or a pharmaceutically acceptable derivative thereof, and a pharmaceutically acceptable carrier therefore.

As used herein the term ‘pharmaceutically acceptable’ embraces compounds, compositions and ingredients for both human and veterinary use: for example the term ‘pharmaceutically acceptable salt’ embraces a veterinary acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged, for instance a pharmaceutical composition, which can be topically applied on the zone of the body which was exposed to cnidaria envenomation.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

Suitable dosages of the vanilloid receptor antagonist include the known doses for these compounds as described or referred to in reference texts such as the British and US Pharmacopoeias, Remington's Pharmaceutical Sciences (Mack Publishing Co.), Martindale The Extra Pharmacopoeia (London, The Pharmaceutical Press) (for example see the 31 st Edition page 341 and pages cited therein) or the above mentioned publications or doses which can be determined by standard procedures. The solid oral compositions may be prepared by conventional methods of blending, filling or tabletting. Repeated blending operations may be used to distribute the active agent throughout those compositions employing large quantities of fillers. Such operations are of course conventional in the art. The Tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration. Compositions may, if desired, be in the form of a pack accompanied by written or printed instructions for use.

The compositions are formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

The invention is further illustrated by way of the illustrative embodiments described below.

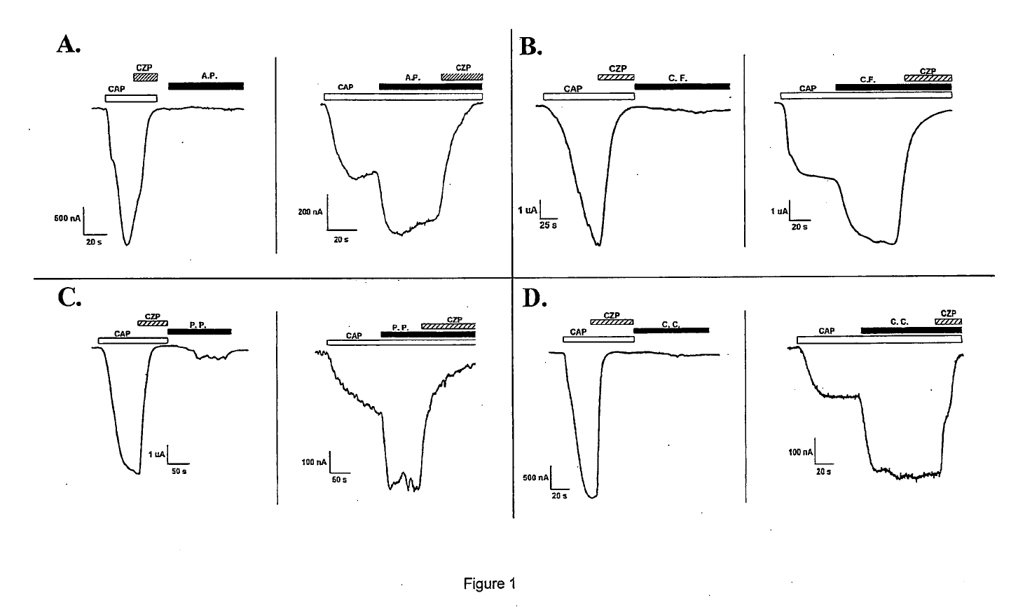

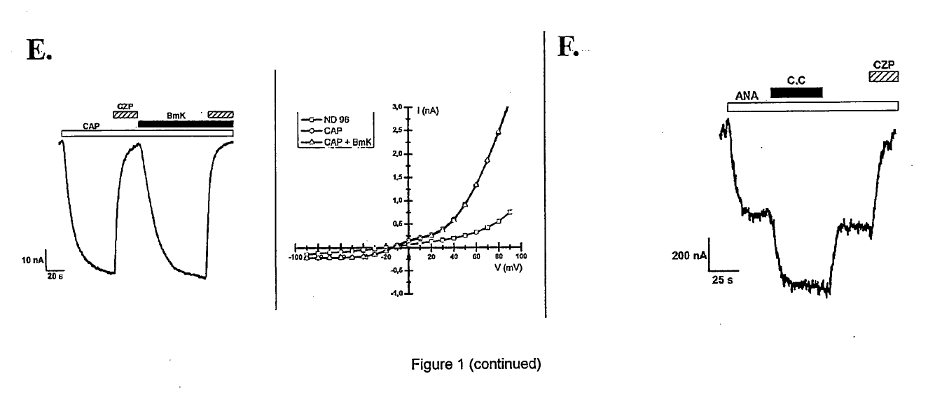

TRPV1 was expressed in Xenopus laevis oocytes and studied with the two-electrode voltage-clamp technique. Currents were measured in ND96 solution using a protocol of −90 mV during 400 s. IV curves were taken with a standard step protocol using a series of 400 ms step pulses from −90 to +90 mV. Temperature and pH were kept respectively at 22° C. and 7.4. As previously described', capsaicin (2 μM) was used as an agonist and capsazepine (10 μM) as an antagonist of TRPV1. We tested crude venom of four cnidarian species (Aiptasia pulchella, Chironex fleckeri, Physalia physalis and Cyanea capillata), each representing one of the four classes in which the phylum Cnidaria is divided (Anthozoa, Cubozoa or sea wasps, Hydrozoa and Scyphozoa or jellyfishes). Although none of the four venoms tested had any significant effect of its own (FIG. 1A-D left), there was a clear allosteric effect on the [(n+1) with (n>0)] activation of the channel when the venom was applied together with capsaicin (FIG. 1A-D right). Because capsaicin is obviously not present at in vivo envenomations, we tested whether an endogenous activator, like anandamide^{13,14}, is also able to give the same allosteric effect together with the venom. When anandamide (10 μM) was applied instead of capsaicin, the same allosteric effect was indeed found (FIG. 1F), indicating that this mechanism might also be operative at in vivo envenomations. Because stings of the scorpion Buthus martensi Karsch (BmK), a very well-studied preparation, are also accompanied with symptoms as redness and burning Pain^{15,16}, we compared the effect of crude BmK venom under the same conditions. This venom did not have any effect on TRPV1 (FIG. 1E), indicating that the allosteric effect might be specific for cnidarian venoms. Crude venom of the green mamba (Dendroapsis angusticeps) also did not have any effect on TRPV1 (data not shown).

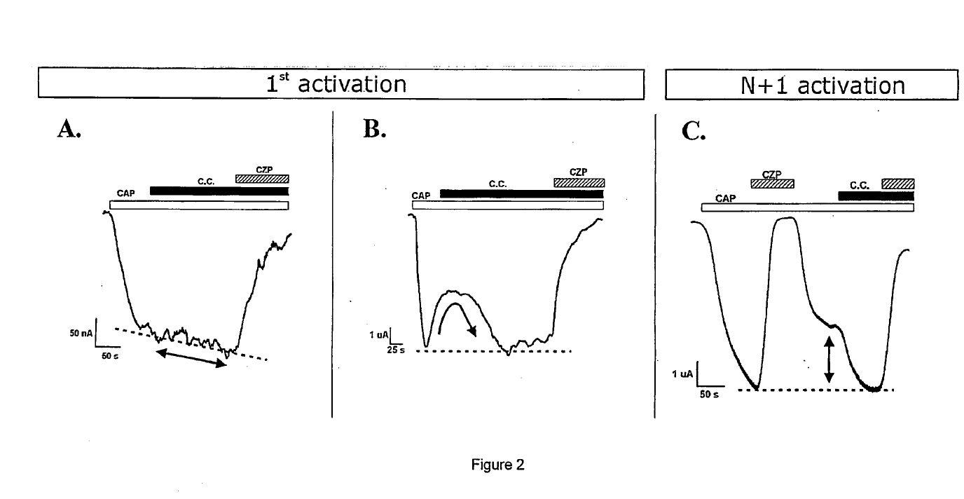

To clarify the mechanism of action of the venom and to investigate whether the observed venom effects were due to desensitization of TRPV1^{19,20}, the effect of the venom on the peak current from the first and consecutive activations was tested. By applying the venom on the peak current, we ensured that there is virtually no desensitization of the channel and if the venom effect is indeed linked to desensitization, there should be no apparent effect of the venom on the peak current. If the application of the venom is performed after the desensitization process has significantly started or on the (n+1) activation of the TRPV1 channel, a desensitization-dependent venom effect was expected. The results of this hypothesis show us that the venom is indeed without apparent effect during a first activation peak of TRPV1 (FIG. 2A). In contrast, the allosteric effect in which the desensitization dependent component is being restored by the venom is clearly present when the venom is given during the desensitization phase or at the (n+1) activation (FIGS. 2B and 2C). As such, these findings clearly indicate that knocking down the desensitization of TRPV1 is related to the venom effect resulting in a larger inward currents that can generate in turn the typical persisting burning pain sensation. This mechanism is of great interest in the further understanding and development of proper treatments of cnidaria envenomations.

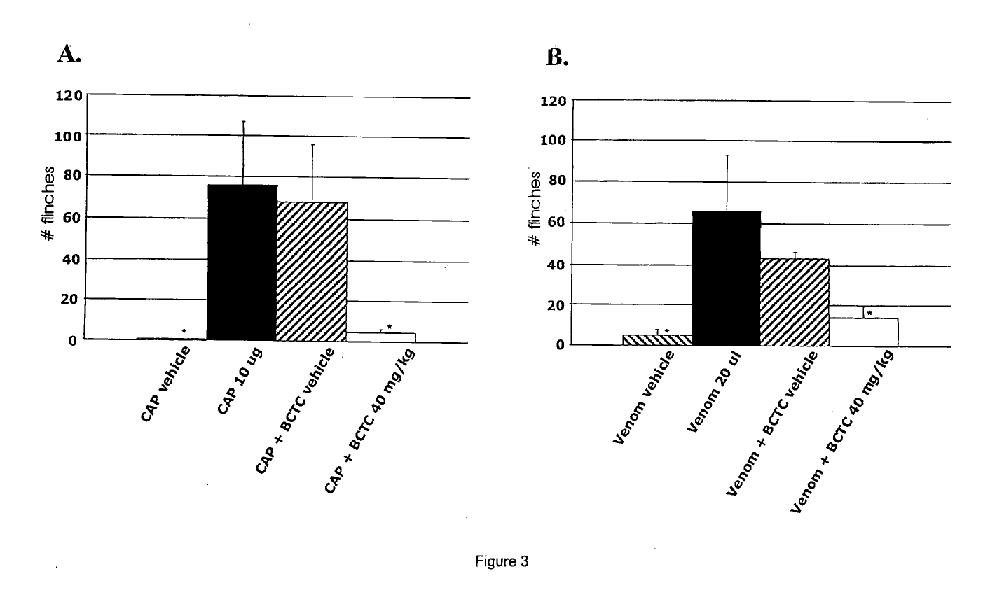

To test the in vivo effect of cnidarian venoms and the possible therapeutic use of TRPV1 antagonists, we performed studies in rats. The number of flinches was counted for 4 minutes after intraplantar injection of capsaicin or venom. The dose of crude Cyanea capillata venom injected was chosen based on the number of flinches that was comparable with the number of flinches seen with 10 μg capsaicin. FIGS. 3A and 3B show there is a significant increase of flinches when compared with the reaction after intraplantar injection of vehicle. Forty mg/kg 4-(3-trifluoromethylpyridin-2-yl)piperazine-1-carboxylic acid (5-trifluoromethylpyridin-2-yl)amide (BCTC), a high-affinity TRPV1 antagonist²¹, was injected subcutaneously one hour before intraplantar injection of capsaicin or Cyanea capillata venom to test whether a TRPV1 antagonist can inhibit their induced pain reaction. FIG. 3 A shows a significant decrease (mean from 76.0 to 4.2, p<0.05, n=6) in pain reaction when rats were injected with capsaicin, following BCTC conditioning. BCTC also gave a significant decrease (mean from 65.8 to 14.0, p<0.05, n=6) in number of flinches upon intraplantar injection of crude venom (FIG. 3B). Control injection of BCTC vehicle did not significantly change the reaction after intraplantar capsaicin or venom injection. The dose needed to decrease half of the number of flinches induced by the venom ranged between 10-40 mg/kg (data not shown). The analgesic effect of BCTC was maximal at a dose of 40 mg/kg as there was no further decrease in flinches when a higher dose was injected. In all cases (capsaicin and venom) there was a rest effect of a few flinches in 4 minutes. This might indicate that other channels are also involved in the pain induction although the relative role thereof will be very small knowing that the induced pain effect is already reduced with 79% by BCTC, a selective TRPV1 antagonist. Taken together, these in vivo results provide supportive evidence that TRPV1 antagonists as analgesics in cnidaria envenomations warrant clinical trials.

In conclusion, we identified TRPV1 as a key component in the signal-transduction pathway of cnidaria envenomation. These newfound data provide a pathophysiologic basis for symptomology of cnidaria stings. Although the active substance(s) in these venoms is (are) not fully identified, our discovery provides important insights into designing more effective treatments for cnidaria envenomation: on the one hand, TRPV1 blockers can possibly be used as therapeutics just as atropine is used as an antidote for organophosphate-type envenomations on the muscarinic ACh receptor²²; on the other hand, one might speculate that TRPV1 activators may also be useful in the treatment of cnidaria stings as they could counteract the venom induced down regulation of the desensitization. As a consequence, an inverse relationship exists between the degree of desensitization and the size of the inward currents and as a corollary hereof the burning pain sensation will diminish once the inward currents get smaller.

### Methods

### Materials

Electrophysiology. Tentacles were used from Aiptasia pulchella, Cyanea capillata, Physalia physalis and Chironex fleckerii. After collection the tentacles were freeze-dried and kept in refrigerator.

In vivo studies. Male Sprague Dawley rats (Harlan, The Netherlands), weighting between 270 and 310 g, were used in in vivo studies.

### Preparation of Samples for Assay.

Tentacles were cut into smaller pieces and suspended in 50-60 ml 10% acetic per 1.5-2 g of tentacles. The mixture was stirred overnight at room temperature with a magnetic stirrer. The sample was centrifuged at 40,000 g for one hour. The supernatant was recovered by careful decantation or removed by a syringe. The sample does not always remain frozen during the freeze-drying from 10% acetic acid, but does so in 1-2% acetic acid. Acetic acid eliminates mucuous material which may clog chromatographic columns. The supernatant was, therefore, concentrated by rotatory evaporation to about 10% of its original volume, diluted with 5-6 volumes of water and freeze-dried. Freeze dried samples were dissolved again in ND96. Solutions were titrated to pH 7.4 or 5.4 with NaOH.

### Electrophysiological Recordings

cRNA transcripts were synthesized from XbaI-linearized VR1 cDNA templates using T7 RNA polymerase (Ambion). The harvesting of oocytes from anaesthetized female Xenopus laevis frogs was performed as previously described²³. Oocytes were injected with 0.5-5 ng TRPV1 cRNA. Two to seven days after injection, two-electrode voltage-clamp recording was performed (E_{hold}=−90 mV). Current-voltage (IV) curves were taken using a series of 400 ms step pulses from −90 to +90 mV. The recording chamber was perfused at a rate of 2 ml with a ND-96 solution containing (in mM) 96 NaCl, 2 KCl, 1.8 CaCl₂, 1 MgCl₂, 5 HEPES, pH 7.4. Temperature of the perfusate was controlled using a SC-20 dual in-line heater/cooler (Warner Instruments). Capsaicin and capsazepine were purchased from Sigma, anandamide from Tocris.

### REFERENCES

1. Burnett, J. W. & Calton, G. J. Venomous pelagic coelenterates: chemistry, toxicology, immunology and treatment of their stings. Toxicon 25, 581-602 (1987).
2. Walker, M. J. Pharmacological and biochemical properties of a toxin containing material from the jellyfish, Cyanea capillata. Toxicon 15, 3-14 (1977).
3. Walker, M. J. The cardiac actions of a toxin-containing material from the jellyfish, Cyanea capillata. Toxicon 15, 15-27 (1977).
4. Yoshimoto, C. M. & Yanagihara, A. A. Cnidarian (coelenterate) envenomations in Hawaii improve following heat application. Trans R Soc Trop Med Hyg 96, 300-3 (2002).
5. Bailey, P. M., Little, M., Jelinek, G. A. & Wilce, J. A. Jellyfish envenoming syndromes: unknown toxic mechanisms and unproven therapies. Med J Aust 178, 34-7 (2003).
6. Kumar, S., Miranda-Massari, J. R., Gonzalez, M. J. & Riordan, H. D. Intravenous ascorbic acid as a treatment for severe jellyfish stings. P R Health Sci J 23, 125-6 (2004).
7. Caterina, M. J. et al. The capsaicin receptor: a heat-activated ion channel in the pain pathway. Nature 389, 816-24 (1997).
8. Clapham, D. E. TRP channels as cellular sensors. Nature 426, 517-24 (2003).
9. Voets, T., Talavera, K., Owsianik, G. & Nilius, B. Sensing with TRP channels. Nat Chem Biol 1, 85-92 (2005).
10. Numazaki, M. & Tominaga, M. Nociception and TRP Channels. Curr Drug Targets CNS Neurol Disord 3, 479-85 (2004).
11. Patapoutian, A. TRP Channels and Thermosensation. Chem. Senses 30 Suppl 1, i193-i194 (2005).
12. Voets, T. et al. The principle of temperature-dependent gating in cold- and heat-sensitive TRP channels. Nature 430, 748-54 (2004).
13. Smart, D. & Jerman, J. C. Anandamide: an endogenous activator of the vanilloid receptor. Trends Pharmacol Sci 21, 134 (2000).
14. Van Der Stelt, M. & Di Marzo, V. Endovanilloids. Putative endogenous ligands of transient receptor potential vanilloid 1 channels. Eur J Biochem 271, 1827-34 (2004).
15. Polis, G. A. The biology of scorpions (Stanford university press, Stanford, 2002).
16. Otero, R. et al. Scorpion envenoming in two regions of Colombia: clinical, epidemiological and therapeutic aspects. Trans R Soc Trop Med Hyg 98, 742-50 (2004).
17. Tominaga, M. & Tominaga, T. Structure and function of TRPV1. Pflugers Arch 451, 143-50 (2005).
18. Carrette, T. J., Cullen, P., Little, M., Peiera, P. L. & Seymour, J. E. Temperature effects on box jellyfish venom: a possible treatment for envenomed patients? Med J Aust 177, 654-5 (2002).
19. Mohapatra, D. P. & Nau, C. Desensitization of capsaicin-activated currents in the vanilloid receptor TRPV1 is decreased by the cyclic AMP-dependent protein kinase pathway. J Biol Chem 278, 50080-90 (2003).
20. Numazaki, M. et al. Structural determinant of TRPV1 desensitization interacts with calmodulin. Proc Natl Acad Sci USA 100, 8002-6 (2003).
21. Swanson, D. M. et al. Identification and biological evaluation of 4-(3-trifluoromethylpyridin-2-yl)piperazine-1-carboxylic acid (5-trifluoromethylpyridin-2-yl)amide, a high affinity TRPV1 (VR1) vanilloid receptor antagonist. J Med Chem 48, 1857-72 (2005).
22. Bajgar, J. Organophosphates/nerve agent poisoning: mechanism of action, diagnosis, prophylaxis, and treatment. Adv Clin Chem 38, 151-216 (2004).
23. Liman, E. R., Tytgat, J. & Hess, P. Subunit stoichiometry of a mammalian K+ channel determined by construction of multimeric cDNAs. Neuron 9, 861-71 (1992).

TABLE 1
molecular structures of compounds known to have TRPV1 antagonistic activity

| | |
|---|---|
| 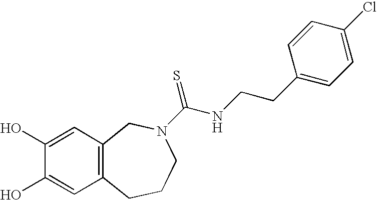 | capsazepine |
| 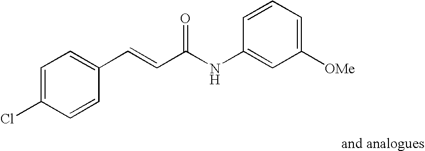 | SB-366791 |
| 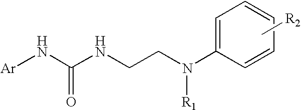 | |
| 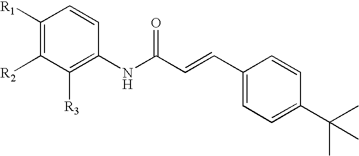 | Anilin analogues |
| 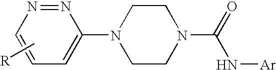 | Pyridazinyl- piperazine analogues |
| 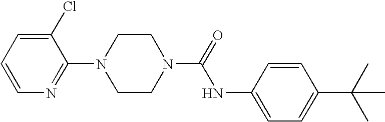 | BCTC |
| 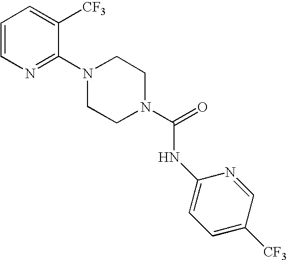 | 4-(3- trifluoromethyl- pyridin- 2- yl)piperazine-1- carboxylic acid (5- trifluoromethyl- pyridin- 2- yl)amide |
| 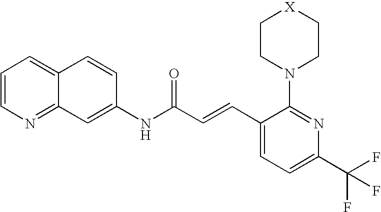 | N-aryl cinnamides Preferably: X = O or X = CH₂ |
| 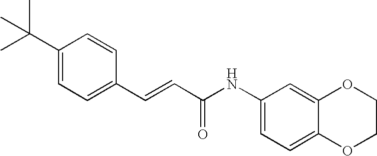 | AMG9810 |
| 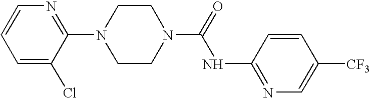 | |
| 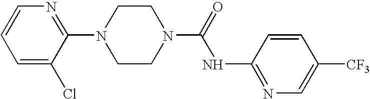 | |
| 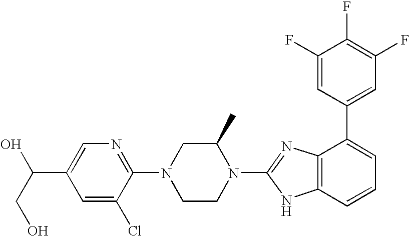 | |
| 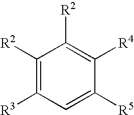 | |
| 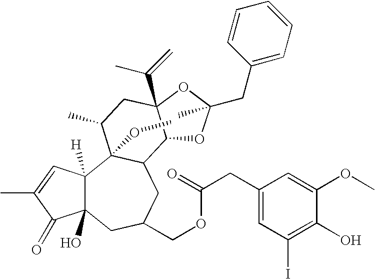 | Iodo-RTX |
| 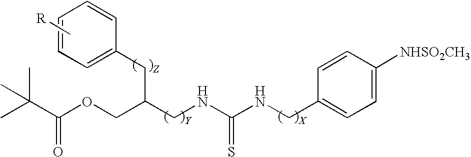 | |
| 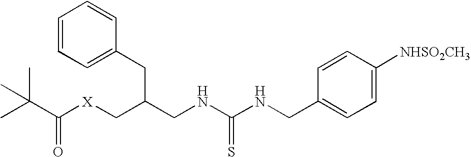 | |
| 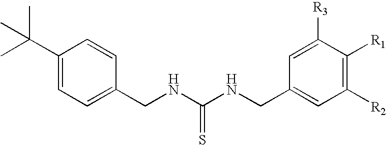 | In particular embodiments: R1 = F and R2 = NHSO₂CH₃ OR R1 = CH₃ and R2 = NHSO₂CH₃ |
| 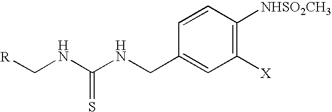 | Journal of medicinal chemistry, 2005, 48, 5823-5836 |
| 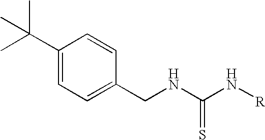 | |
| 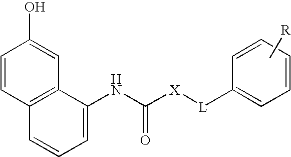 | |
| 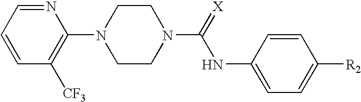 | |
| 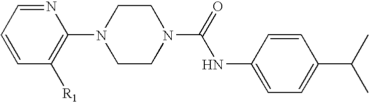 | |
| 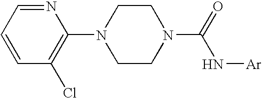 | |
| 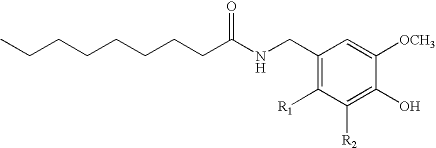 | Preferably, R1 = H, R2 = Cl, Br, I OR R1 = Cl, Br, I and R2 = H |
| 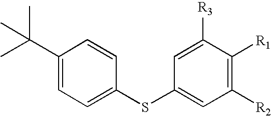 | |
| 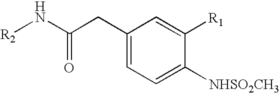 | |
| 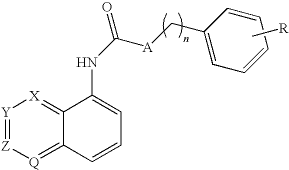 | Journal of medicinal chemistry, 2005, 48, 744-752 |
| 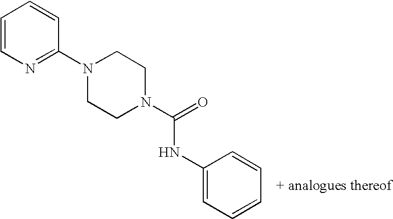 | Journal of medicinal chemistry, 2005, 48, 1857-1872 |
| 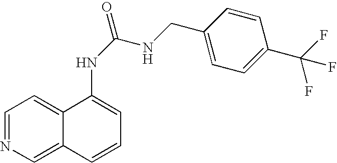 | A-425619 |
| 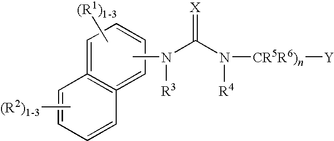 | |
| 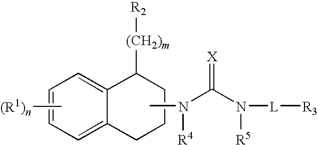 | |
| 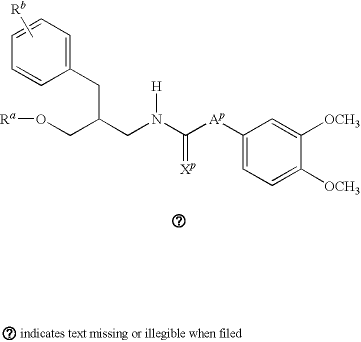 | |
| 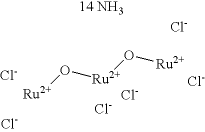 | Ruthenium red |
| 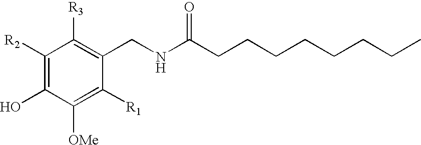 | Journal of medicinal chemistry, 2005, 48, 4663-4669 |
| 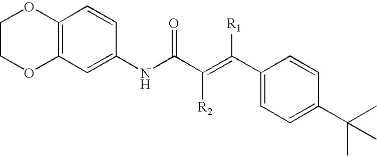 | Journal of medicinal chemistry, 2005, 48, 71-90 |
| 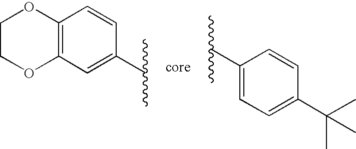 | Journal of medicinal chemistry, 2005, 48, 71-90 |
| 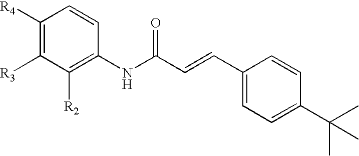 | Journal of medicinal chemistry, 2005, 48, 71-90 |
| 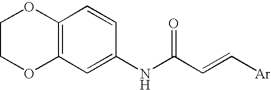 | Journal of medicinal chemistry, 2005, 48, 71-90 |
| 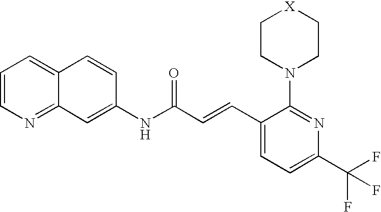 | Journal of medicinal chemistry, 2005, 48, 71-90 |
| 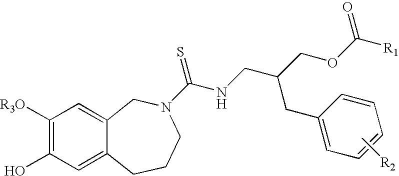 | Preferably R1 = Ph, R2 = H and R3 = H Bioorganic & medicinal chemistry letters 9 (2001) 1713-1720 |
| 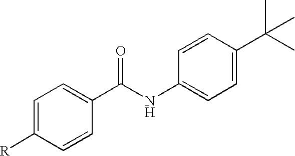 | Bioorganic & medicinal chemistry letters 15 (2005) 631-634 |
| 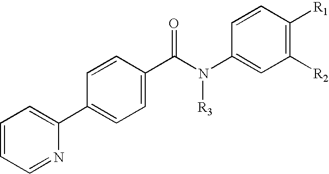 | Bioorganic & medicinal chemistry letters 9 (2001) 1713-1720 |
| 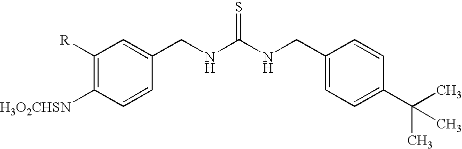 | |
| 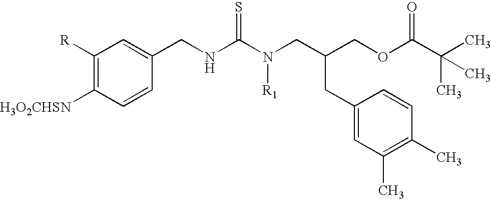 | |
| 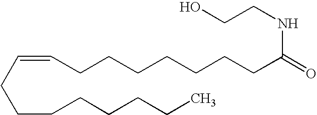 | oleoylethanolamide |
| 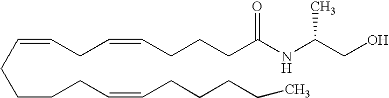 | methanadamide |
| 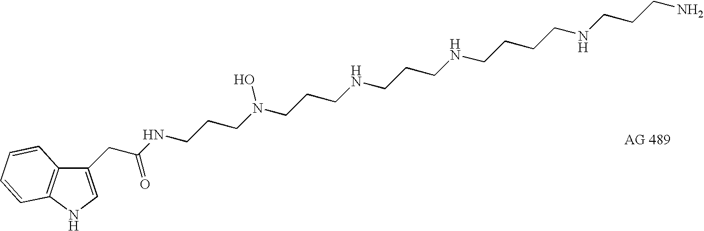 | |
| 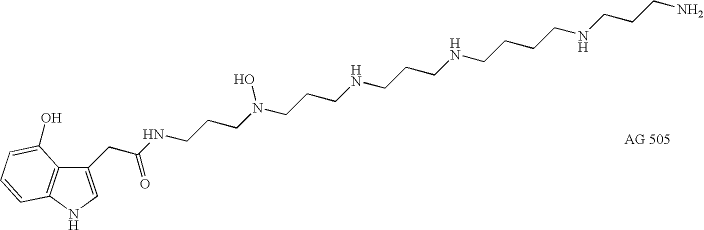 | |
| 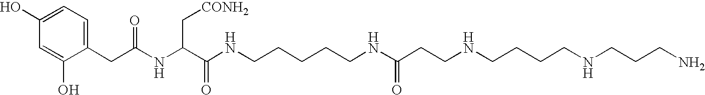 | JSTx-3 |
| 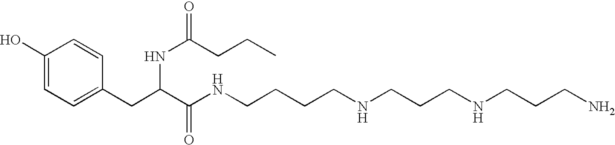 | PhTx = 33 |
| 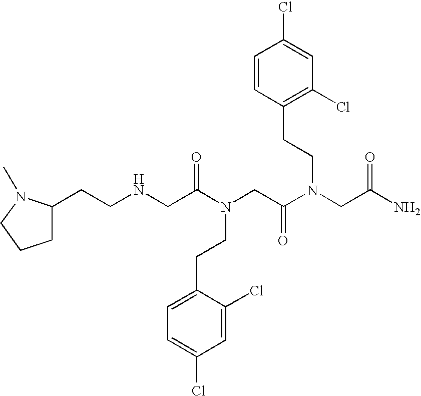 | DD161515 |
| 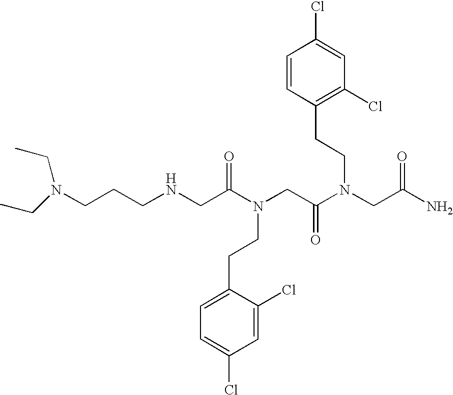 | DD191515 |
| 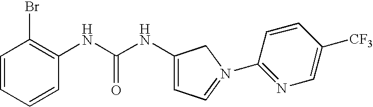 | SB-705498 |
| 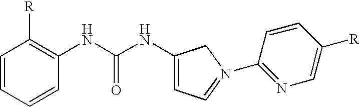 | |
| 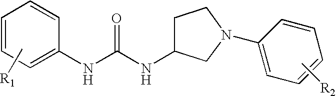 | |
| 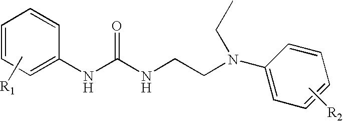 | |
| 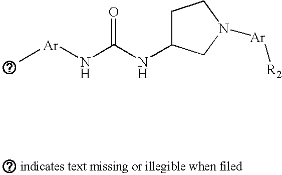 | |
| 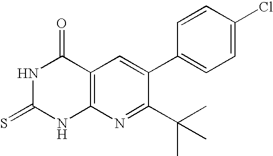 | |
| 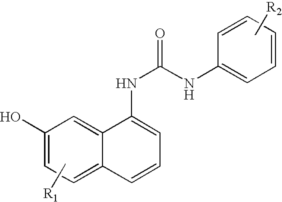 | |
| 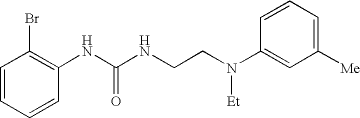 | SB-452533 |
| 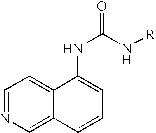 | 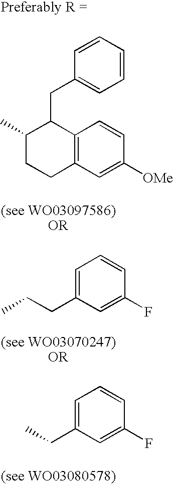 |
| 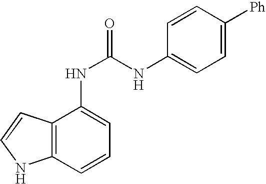 | (see WO030055484) |
| 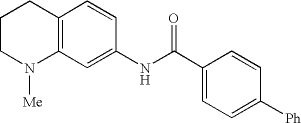 | (see WO03068749) |
| 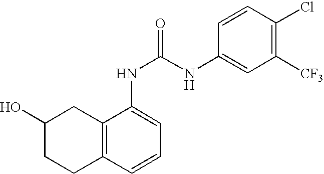 | (see WO03095420) |
| 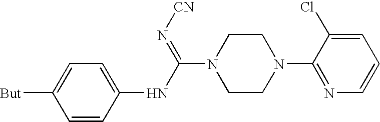 | (see WO04002983) |
| 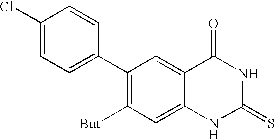 | (see WO02076946) |
| 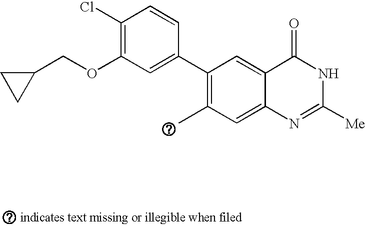 | (see WO03062209) |
| 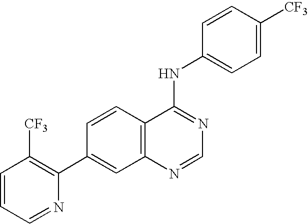 | |
| 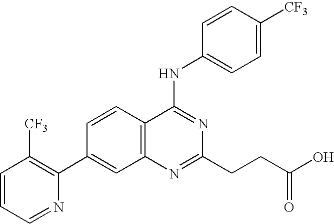 | (see WO04055004) |
| 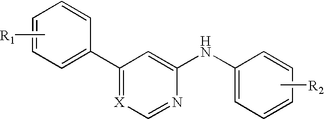 | X = C or N (see WO003049702, WO03099284) |
| 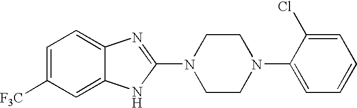 | (see WO004035549) |
| 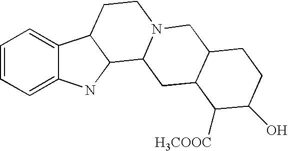 | Yohimbine and deriviatives |
| 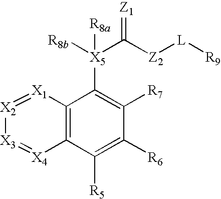 | |
| 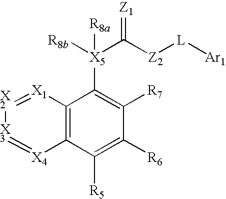 | |
| 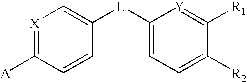 | |
| 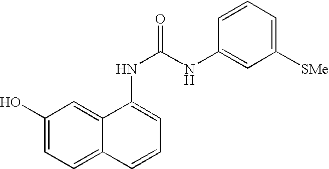 | |
| 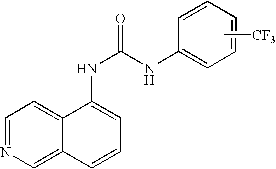 | |
| 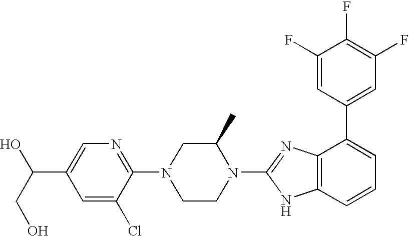 | |
| 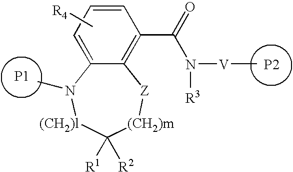 | P1 and P2 can either be similar or different ring structures |
| 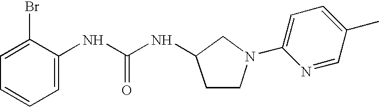 | |
| 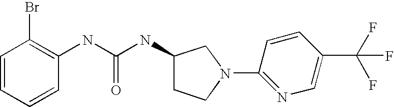 | |
| 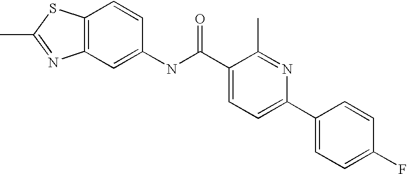 | |
| 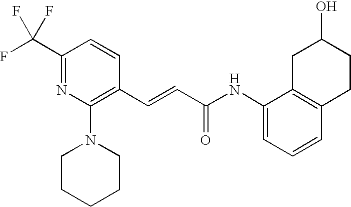 | |
| 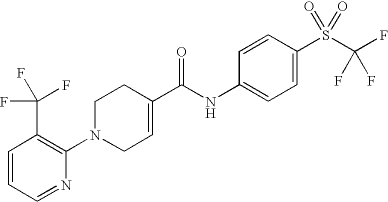 | |
| 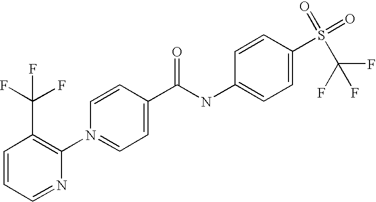 | |
| 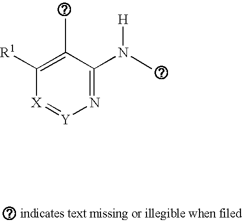 | |
| 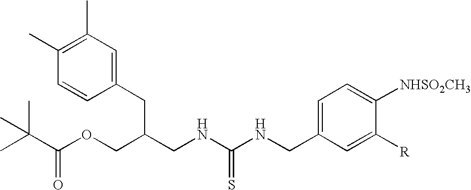 | Preferably R = H OR R = F |
| 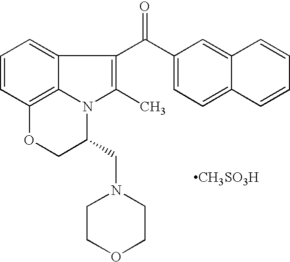 | WIN 55,212-2 |
| 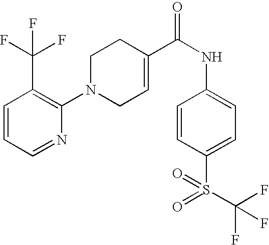 | A-784168 |
| 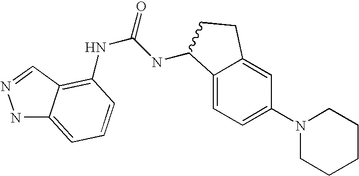 | A-795614 |
| 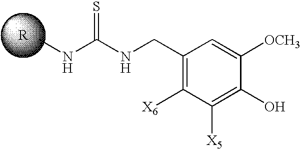 | Bioorganic & Medicinal Chemistry Letters (2007) 214-219 |
| 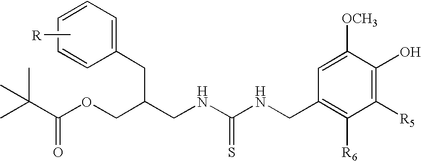 | Bioorganic & Medicinal Chemistry Letters (2007) 214-219 |
| 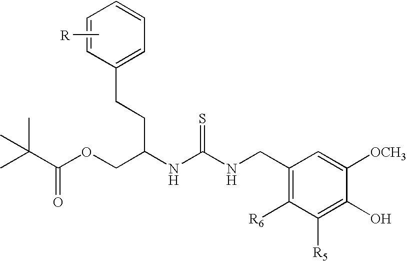 | Bioorganic & Medicinal Chemistry Letters (2007) 214-219 |
| 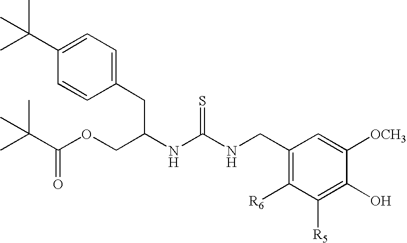 | Bioorganic & Medicinal Chemistry Letters (2007) 214-219 |
| 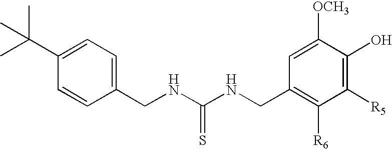 | Bioorganic & Medicinal Chemistry Letters (2007) 214-219 |
| 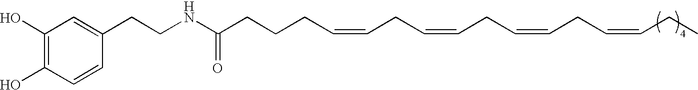 | NADA |
| 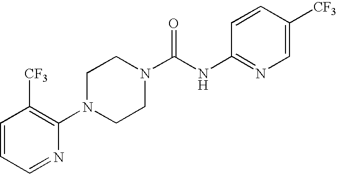 | JNJ-17203212 |
| 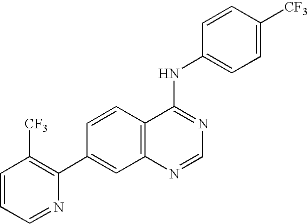 | Quinazoline analogue |
| 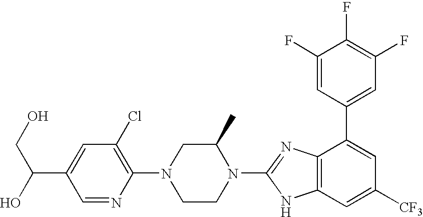 | 46ad (benzimidazool analogue) |
| 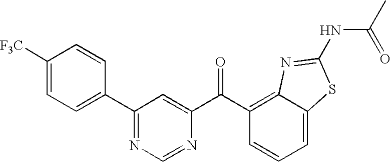 | AMG517 |

1-27. (canceled)

28. The use of a vanilloid receptor antagonist or a pharmaceutically acceptable derivative thereof, in the manufacture of a medicament for the treatment and/or prophylaxis of cnidaria envenomations and the pain associated therewith.

29. The use of a vanilloid receptor antagonist according to claim 28 wherein said antagonist is a vanilloid receptor 1 antagonist.

30. The use of a vanilloid receptor 1 antagonist according to claim 29 wherein said vanilloid receptor 1 antagonist is a derivative of capsaicin, resiniferatoxin, nordihydrocapsaicin, nonivamide, arvanil and phenacetylrinvanil.

31. The use of a vanilloid receptor 1 antagonist according to claim 30 wherein said vanilloid receptor 1 antagonist is capsazepine, iodo-resiniferatoxin or a derivative thereof.

32. The use of a vanilloid receptor 1 antagonist according to claim 30 wherein said vanilloid receptor 1 antagonist is a halogenated derivative of nordihydrocapsaicin, nonivamide, arvanil or phenacetylrinvanil.

33. The use of a vanilloid receptor 1 antagonist according to claim 29 wherein said vanilloid receptor 1 antagonist is a fused azabicyclic compound.

34. The use of a vanilloid receptor 1 antagonist according to claim 29 wherein said vanilloid receptor 1 antagonist is a fused heterocyclic compound.

35. The use of a vanilloid receptor 1 antagonist according to claim 29 wherein said vanilloid receptor 1 antagonist is a fused pyridine compound.

36. The use of a vanilloid receptor 1 antagonist according to claim 29 wherein said vanilloid receptor 1 antagonist is an urea or thiourea derivative.

37. The use of a vanilloid receptor 1 antagonist according to claim 36 wherein said vanilloid receptor 1 antagonist is a pyridyl piprazyl urea derivative.

38. The use of a vanilloid receptor 1 antagonist according to claim 36 wherein said vanilloid receptor 1 antagonist is a beta-aminotetralin-urea derivative.

39. The use of a vanilloid receptor 1 antagonist according to claim 36 wherein said vanilloid receptor 1 antagonist is a heteroaromatic urea derivative.

40. The use of a vanilloid receptor 1 antagonist according to claim 36 wherein said vanilloid receptor 1 antagonist is an isoquinoline urea derivative.

41. The use of a vanilloid receptor 1 antagonist according to claim 36 wherein said vanilloid receptor 1 antagonist is an arylurea or biarylurea derivative.

42. The use of a vanilloid receptor 1 antagonist according to claim 29 wherein said vanilloid receptor 1 antagonist is a cinnamide derivative.

43. The use of a vanilloid receptor 1 antagonist according to claim 29 wherein said vanilloid receptor 1 antagonist is an arginine rich peptide, a ginsenoside, a aminoquinazoline or a Terpen.

44. The use of a vanilloid receptor 1 antagonist according to claim 29 wherein said vanilloid receptor 1 antagonist is selected out of the group consisting of isovelleral, N-arachidonoyl-serotonin, oleoylethanolamide, Methanandamide and derivatives thereof.

45. The use of a vanilloid receptor 1 antagonist according to claim 29 wherein said vanilloid receptor 1 antagonist is a besylate salts of a six-membered amino-heterocycle.

46. A method for the treatment and/or prophylaxis of cnidaria envenomations and the pain associated therewith, said method comprising the administration of an effective, non-toxic and pharmaceutically acceptable amount of a vanilloid receptor antagonist.

47. The method according to claim 46 wherein said antagonist is a vanilloid receptor 1 antagonist.

48. The method according to claim 47 wherein said vanilloid receptor 1 antagonist is a derivative of capsaicin, resiniferatoxin, nordihydrocapsaicin, nonivamide, arvanil and phenacetylrinvanil.

49. A pharmaceutical composition for the treatment and/or prophylaxis of cnidaria envenomations and the pain associated therewith, which composition comprises a vanilloid receptor antagonist or a pharmaceutically acceptable derivative thereof.

50. A pharmaceutical composition according to claim 49 wherein said vanilloid receptor antagonist is a vanilloid receptor 1 antagonist.

51. A pharmaceutical composition according to claim 50 wherein said vanilloid receptor 1 antagonist is a derivative of capsaicin, resiniferatoxin, nordihydrocapsaicin, nonivamide, arvanil and phenacetylrinvanil.

52. The use of a vanilloid receptor antagonist or a pharmaceutically acceptable derivative thereof in the treatment and/or prophylaxis of cnidaria envenomations and the pain associated therewith.

53. The use of a vanilloid receptor antagonist according to claim 52 wherein the vanilloid receptor antagonist is a vanilloid receptor 1 antagonist.

54. The use of a vanilloid receptor antagonist according to claim 53 wherein said vanilloid receptor 1 antagonist is a derivative of capsaicin, resiniferatoxin, nordihydrocapsaicin, nonivamide, arvanil and phenacetylrinvanil.
